(19) United States
(12) Patent Application Publication
(10) Pub. No.: US 2025/0185928 A1
(43) Pub. Date: Jun. 12, 2025

(54) IMPLANTABLE DEVICE AND METHOD FOR DETECTING IN VIVO PRESSURE IN AN ORGAN OF A SUBJECT

(71) Applicant: IOP MEDICAL LTD., Katzrin (IL)

(72) Inventor: Mark ISHAY, Haifa (IL)

(21) Appl. No.: 18/844,966

(22) PCT Filed: Mar. 8, 2023
(86) PCT No.: PCT/IL2023/050240

§ 371 Date: Sep. 8, 2024

(30) Foreign Application Priority Data
Mar. 8, 2022 (IL) 291216

(51) Int. Cl. A61B 5/0215; A61B 3/16; A61B 5/03

(52) U.S. Cl. CPC A61B 5/0215; A61B 3/16; A61B 5/03

(57) ABSTRACT

An implantable device is disclosed, comprising: a tube having a first internal lumen comprising one or more microparticles surrounded by a first liquid; a liquid reservoir comprising a flexible cover, in fluid connection with a first end of the first internal lumen, for providing a second liquid to the first internal lumen; a gas reservoir in fluid connection with a second end of the first internal lumen; wherein the microparticles comprise a material configured to be detected by an external device. In some embodiments, the first liquid and the second liquid are the same liquid. In some embodiments, wherein the first liquid and the second liquid are unmixable. In some embodiments, each microparticle has a diameter between 0.5 to 0.9 of the first internal lumen's hydraulic diameter and comprises a material allowing detection of a movement of each microparticle separately by an external device.

### CROSS-REFERENCE TO RELATED APPLICATIONS

This application claims the benefit of priority of Israeli Patent Application No. 291216, filed Mar. 8, 2022, and entitled: “IMPLANTABLE DEVICE AND METHOD FOR DETECTING IN VIVO PRESSURE IN AN ORGAN OF A SUBJECT” which is hereby incorporated by reference in its entirety.

### FIELD OF THE INVENTION

The present invention relates generally to implantable devices and methods for detecting in vivo pressure in an organ of a subject. More specifically, the present invention relates to implantable devices and methods for detecting in vivo pressure in an organ of a subject using microparticles.

### BACKGROUND OF THE INVENTION

Measuring in vivo pressure in an organ of a subject, for example, measuring intraocular pressure in the eye may allow early detection of diseases, such as, Glaucoma, high blood pressure, and the like.

Glaucoma is an eye disease associated with the damage of the optic nerve leading to a progressive and irreversible loss of vision. Glaucoma is characterized by the irreversible loss of retinal ganglion cells which eventually leads to blindness. Intraocular pressure (IOP) is the major cause of glaucoma and lowering IOP is the only way to avoid irreversible vision loss. The treatment of glaucoma relies heavily on accurately measuring and monitoring IOP levels.

The currently known methods for detecting Glaucoma include static intraocular pressure measurements and frequent punctual measurements. The major drawback of these methods is that both methods have to be performed by an experienced and skilled operator, are taken during the day (in a clinic) when the pressure in the eye is the lowest. Furthermore, and due to the above, these methods are limited to a single snapshot of the eye and thus are inefficient for checking changes in IOP over hours of the day and night or e.g., on a daily basis.

High blood pressure (Hypertension) is a long-term medical condition in which the blood pressure in the arteries is persistently elevated. Long-term high blood pressure is a major risk factor for stroke, coronary artery disease, heart failure, atrial fibrillation, peripheral arterial disease, vision loss, chronic kidney disease, and dementia.

Blood pressure measurements are conducted at a specific time by a dedicated automatic device or by a professional. Multiple blood pressure readings (at least two) spaced 1-2 minutes apart should be obtained to ensure accuracy. Ambulatory blood pressure monitoring over 12 to 24 hours is the most accurate method to confirm the diagnosis. However, all the methods require the subject to wear a blood pressure cuff to a bare upper arm and measure a discrete measurement each time. The device is very uncomfortable and cannot be worn continuously over the entire life of the subject,

Accordingly, there is a need for implantable devices and methods for detecting in vivo pressure in an organ of a subject, that are both comfortable and can provide pressure measurements over long periods of time (e.g., hours, days, weeks and longer).

### SUMMARY OF THE INVENTION

Some aspects of the invention are directed to an implantable device, comprising: a tube having a first internal lumen comprising one or more microparticles surrounded by a first liquid; a liquid reservoir comprising a flexible cover, in fluid connection with a first end of the first internal lumen, for providing a second liquid to the first internal lumen; a gas reservoir in fluid connection with a second end of the first internal lumen; wherein the microparticles comprise a material configured to be detected by an external device. In some embodiments, the first liquid and the second liquid are the same liquid. In some embodiments, wherein the first liquid and the second liquid are unmixable. In some embodiments, each microparticle has a diameter between 0.5 to 0.9 of the first internal lumen's hydraulic diameter and comprises a material allowing a detection of a movement of each microparticle separately by an external device.

In some embodiments, a density of the microparticles is lower than a density of the first liquid by at least 5%. In some embodiments, the tube is curved or straight. In some embodiments, the tube is made from a flexible material.

In some embodiments, the microparticles include a material visible in an image taken by an optical camera. In some embodiments, the microparticles are colored with a color detectable in the image. In some embodiments, the microparticles are colored with fluorescent color.

In some embodiments, the microparticles include a magnetic material and/or an electrically conductive material. In some embodiments, a diameter of each microparticle is between 0.5 to 0.9 of the first internal lumen's hydraulic diameter.

In some embodiments, wherein the liquid reservoir is a balloon and wherein the flexible cover is included in the outer shell of the balloon. In some embodiments, the implantable device further comprises a second internal lumen being the gas reservoir. In some embodiments, the liquid reservoir has a flat shape with an open face covered by a flexible membrane. In some embodiments, the gas reservoir also has a flat shape.

In some embodiments, the organ is an eye of the subject and wherein the hydraulic diameter of the first inner lumen is between 40 to 150 microns. In some embodiments, the organ is a blood vessel of the subject and wherein the hydraulic diameter of the first inner lumen is between 150 to 3500 microns. In some embodiments, the organ is a lung segment of the subject and wherein the hydraulic diameter of the first inner lumen is between 120 to 1500 microns.

In some embodiments, a material of the flexible cover is selected such that a pressure applied on the flexible cover, after the implantation, causes a flow of liquid from the liquid reservoir into the first internal lumen. In some embodiments, the microparticles are microcapsules.

Some additional aspects of the invention are directed to a method of determining in vivo pressure in an organ of a subject, comprising: receiving from an external detector a signal indicative of a location of one or more microparticles included in an implantable device implanted in the organ; determining the location of the one or more microparticles based on the signal; receiving at least one previously recorded location of the one or more microparticles; detecting a change in the location of the microparticles between the received location and the at least one previously recorded location; and determining the in vivo pressure based on the change in the location.

In some embodiments, determining the location of the one or more microparticles comprises: receiving at least two known locations in the implantable device; identifying the at least two known locations in the received signal; and determining the location of the one or more microparticles based on the signal indicative of the location of the one or more microparticles and the at least two signals.

In some embodiments, receiving at least one previously recorded location of the one or more microparticles comprises receiving a plurality of previously recorded locations associated with different dates. In some embodiments, the organ is an eye and wherein the in vivo pressure is an intraocular pressure. In some embodiments, the external detector is an optical camera, and the method comprises: receiving from the optical camera an image of the eye that includes an image of the one or more microparticles; determining a location of the one or more microparticles in the internal lumen from the image; receiving at least one previously recorded location of the microparticles in the internal lumen; and detecting intraocular pressure if the change in the location is greater than a threshold value.

In some embodiments, the organ is a blood vessel and wherein the in vivo pressure is blood pressure. In some embodiments, the external detector is thermal imaging, X-Ray imagining, magnetic imagining (MRI), and computed tomography (CT).

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A is an illustration of an implantable device for detecting in vivo pressure in an organ of a subject according to some embodiments of the invention;

FIG. 1B is an illustration of another implantable device for detecting in vivo pressure in an organ of a subject according to some embodiments of the invention;

FIG. 1C is an illustration of another implantable device for detecting in vivo pressure in an organ of a subject according to some embodiments of the invention;

FIG. 2 is an illustration of the device implanted in an eye of the subject according to some embodiments of the invention;

FIG. 3 is an illustration of another implantable device for detecting in vivo pressure in an organ of a subject according to some embodiments of the invention;

FIG. 4 is a flowchart of a method of detecting in vivo pressure in an organ of a subject according to some embodiments of the invention; and

FIG. 5 is a block diagram, depicting a computing device that may be included in a system for detecting in vivo pressure in an organ of a subject according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or another electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Some aspects of the invention are directed to devices to be implanted in an organ of a subject, for example, in the eye, a blood vessel, a lung segment, and the like, in order to measure an internal pressure in the organ. Such a device when implanted in the eye may allow a simple and reliable way for detecting early stages of glaucoma. In some embodiments, such a device when implanted in an artery may allow monitoring of blood pressure in patients with a high risk of developing hypertension.

A device according to embodiments of the invention may include at least one tube having an internal lumen comprising one or more microparticles. In some embodiments, the one or more microparticles may include a material configured to be detected by an external device. For example, each microparticles may include a color (e.g., a fluorescent color) detectable in an image taken by a camera. In another example, the one or more microparticles may include a conductive or magnetic material configured to be detected by a thermal camera, a magnetic sensor and the like. In some embodiments, the a material may allow a detection of a movement of at least one microparticle (each microparticle separately) by an external device.

In some embodiments, the internal lumen may include a liquid, having a density higher than the density of the microparticles, surrounding microparticles. The liquid may be provided from a liquid reservoir comprising a flexible cover, connected to one end of the lumen. Accordingly, when pressure is applied on the flexible cover, the liquid from the reservoir may push the microparticles to move inside the internal lumen. In some embodiment, in order to allow changes in the volume occupied by the liquid, a gas reservoir may be connected to the other end of the internal lumen, opposite to the liquid reservoir. In some embodiments, the movement of the microparticles due to the application of the internal pressure (e.g., intraocular pressure in the eye, blood pressure in an artery, etc.) on the liquid reservoir may be indicative/relative to the level of the internal pressure.

Devices according to some embodiments of the invention do not include or require any power source, nor need to be powered. Therefore, such devices can remain in the organ for several years, for example, 5 to 10 years without any intervention.

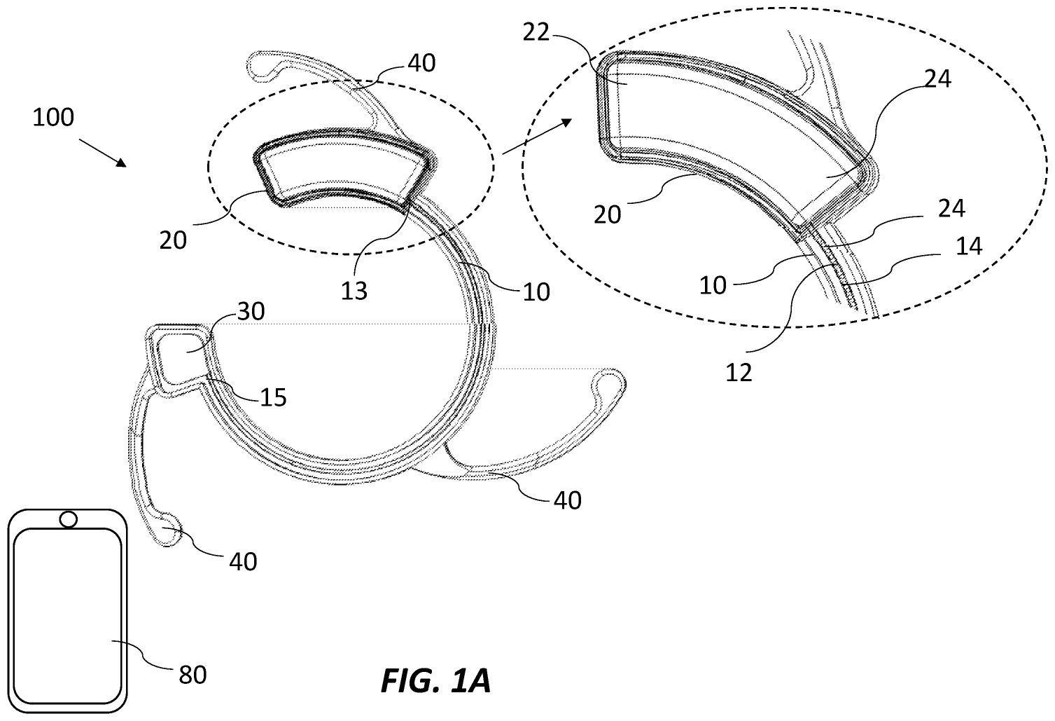

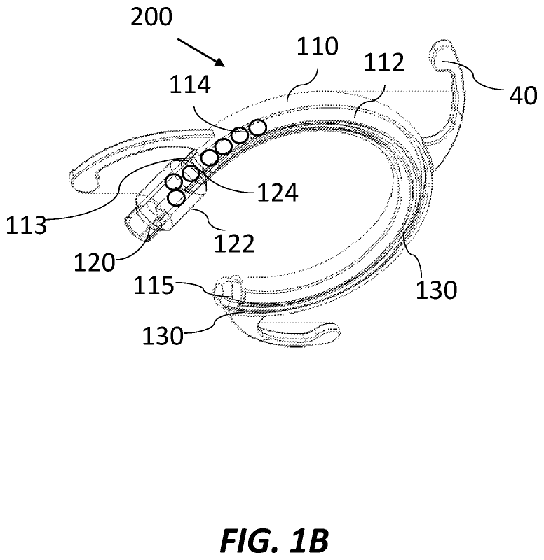

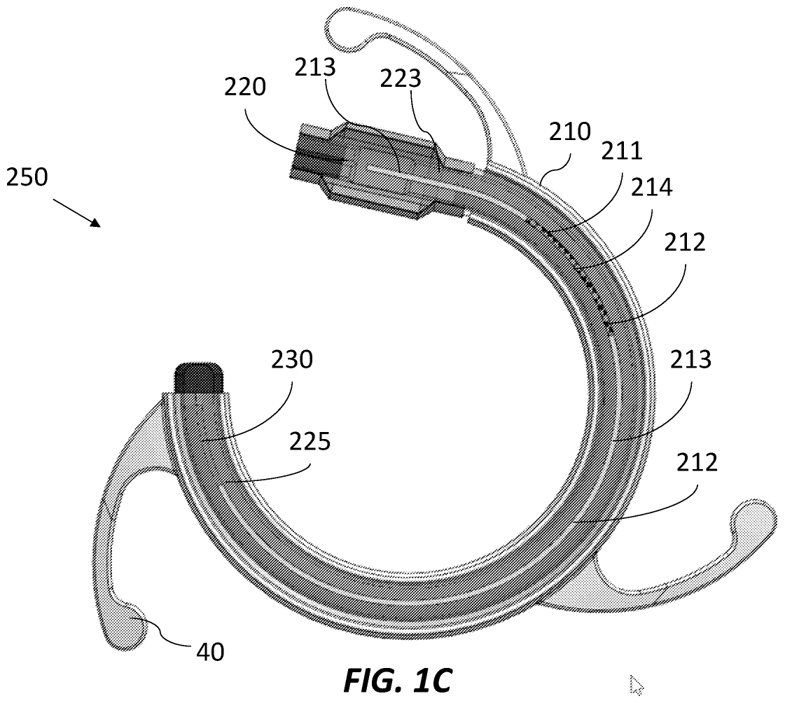

Reference is now made to FIG. 1A which is an illustration of a nonlimiting example for an implantable device according to some embodiments of the invention. An implantable device 100 may include a tube 10 having a first internal lumen 12 comprising microparticles 14 surrounded by a liquid 24 (e.g., a first liquid 211, illustrated in FIG. 1C). In some embodiments, device 100 (and therefore tube 10) may be dimensioned to be implanted in an organ of a subject. In the nonlimiting example, illustrated in FIG. 1A the organ may be the eye of the subject and therefore, a hydraulic diameter of first inner lumen 12 is between 40 to 150 microns.

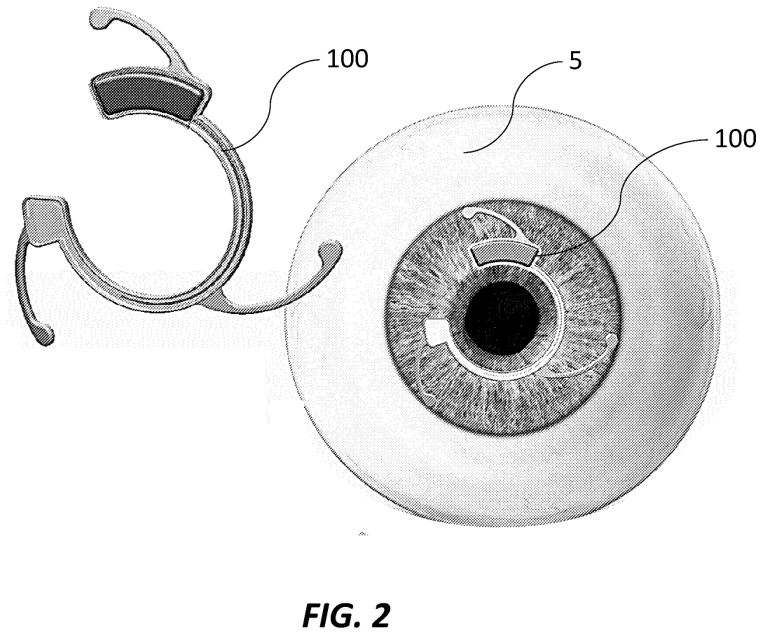

In some embodiments, one or more microparticles 14 may include a material configured to allow the detection of one or more microparticles by an external device. In some embodiments, when the organ is the eye, the external device may be a camera, for example, of a user device 80 taking images of the eye, as discussed and illustrated with respect to FIG. 2. In such cases one or more microparticles 14 include a material visible in an image taken by the optical camera, for example, one or more microparticles 14 may be colored with a color detectable in the image, or microparticles 14 may be colored with fluorescent color.

In some embodiments, the diameter of each microcapsule 14 is between 0.5 to 0.9 of the diameter of first internal lumen 12 hydraulic diameter. In some embodiments, the diameter of each microcapsule 14 is between 0.6 to 0.9 of the diameter of first internal lumen 12 hydraulic diameter. In some embodiments, the diameter of each microcapsule 14 is between 0.5 to 0.8 of the diameter of first internal lumen 12 hydraulic diameter. In some embodiments, the diameter of each microcapsule 14 is between 0.6 to 0.8 of the diameter of first internal lumen 12 hydraulic diameter. In some embodiments, the cross-section of first internal lumen 12 may be circular, elliptical, rectangular, hexagonal, or the like. For example, when the hydraulic diameter of first inner lumen 12 is between 40 to 150 microns, the diameter of each microcapsule 14 is between 20 microns to 135 microns, or any value in between. In some embodiments, the diameter of each microcapsule 14 is larger than 15 micron.

In some embodiments, device 100 may further include a liquid reservoir 20 comprising a flexible cover 22, in fluid connection with a first end 13 of first internal lumen 12, for providing liquid 24 (e.g., a second liquid 213 illustrated in FIG. 1C or the first liquid). In some embodiments, a density of microparticles 14 is lower than the density of liquid 24 by at least 5%, for example, by at least 7%, by at least 10%, by at least 15%, by at least 20%, by at least 30%, by at least 40%, by at least 50% or more. In the nonlimiting example, microparticles 14 may be microcapsules filled with air or any other gas or solution lighter than the liquid, and liquid 24 may be water, hypodermoclysis, any intravenous infusion, silicone-based gel or other viscous biocompatible fluid and the like.

In some embodiments, a material of flexible cover 22 is selected such that a pressure applied on flexible cover 22 (e.g., the intraocular pressure), after the implantation, causes a flow of liquid 24 from liquid reservoir 20 into first internal lumen 12. In the nonlimiting example of FIG. 1A liquid reservoir 20 has a flat shape with an open face covered by flexible cover 22 is a membrane made from any thin biocompatible polymeric material covering liquid 24 inside liquid reservoir 20. Some nonlimiting examples for, biocompatible polymeric material may include liquid crystal polymer (LCP), poly(methyl methacrylate) (PMMA), polysulfone (PSU), polyphenylsulfone (PPSU), and any silicone-based films, include but not limited to Polydimethylsiloxane (PDMS), butyl rubber and the like.

In some embodiments, device 100 may further include a gas reservoir 30 in fluid connection to a second end 15 of first internal lumen 12. In the nonlimiting example of FIG. 1A gas reservoir 30 has a flat shape.

In some embodiments, device 100 may further include one haptic elements 40 for fixating device 100 in the organ, for example, the eye.

Reference is now made to FIG. 1B which is an illustration of another nonlimiting example of an implantable device according to some embodiments of the invention. An implantable device 200 may include a tube 110 having a first internal lumen 112 comprising one or more microparticles 114 surrounded by a liquid 124. In some embodiments, a diameter of each microcapsule 114 is between 0.5 to 0.9 of the first internal lumen's hydraulic diameter. In some embodiments, device 200 may be dimensioned to be implanted in an organ of a subject. In the nonlimiting example, illustrated in FIG. 1B the organ may be the eye of the subject and therefore, a hydraulic diameter of first inner lumen 112 is between 40 to 150 microns. In some embodiments, the diameter of each microcapsule 114 is equal to or larger than 15 microns.

In some embodiments, microparticles 114 may be substantially the same as, microparticles 14 of device 100.

In some embodiments, device 200 may further include a liquid reservoir 120 comprising a flexible cover 122, in fluid connection with a first end 113 of first internal lumen 112, for providing liquid 124 for surrounding microparticles 114. In some embodiments, a density of microparticles 114 is lower than the density of liquid 124 by at least 5%, for example, by at least 7%, by at least 10%, by at least 15%, by at least 20%, by at least 30%, by at least 40%, by at least 50% or more. In a nonlimiting example, microparticles 114 may be microcapsules filled with air or any other gas or solution lighter than the liquid, and liquid 124 may be water, hypodermoclysis, any intravenous infusion, silicone-based gel or other viscous biocompatible fluid and the like.

In some embodiments, a material of flexible cover 122 is selected such that a pressure applied on flexible cover 122 (e.g., the intraocular pressure), after the implantation, causes a flow of liquid 124 from liquid reservoir 120 into first internal lumen 112. In the nonlimiting example of FIG. 1B liquid reservoir 120 is a balloon and the flexible cover 122 is included in the outer shell of the balloon.

In some embodiments, device 200 may further include a gas reservoir 130 in fluid connection to a second end 115 of first internal lumen 112. In the nonlimiting example of FIG. 1B, tube 110 may include a second internal lumen 130 serving as a gas reservoir.

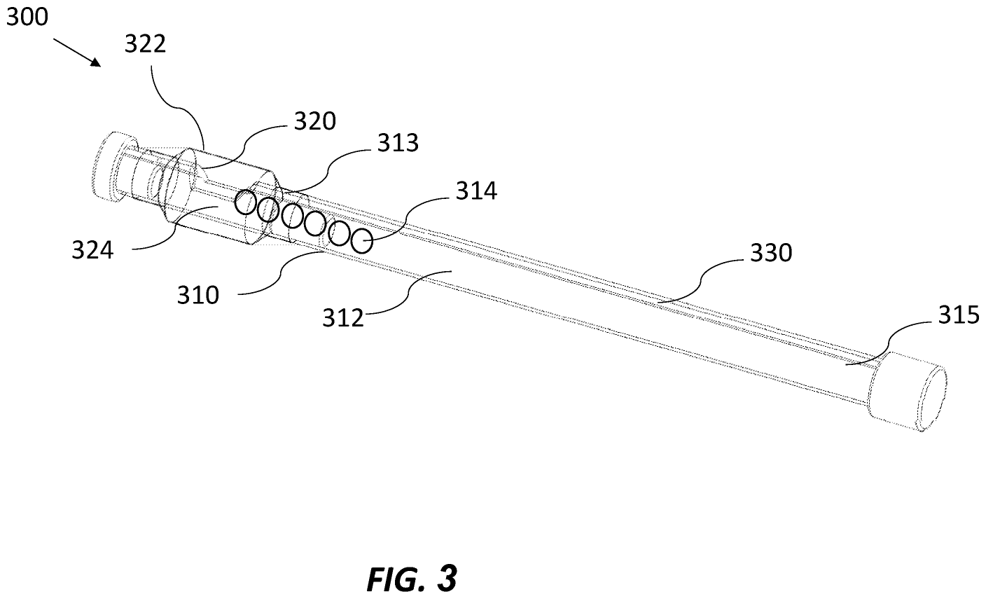

In some embodiments, tubes 10 (in FIG. 1A) or 110 (in FIG. 1B) are curved. According to other embodiments, tube 310 is substantially straight as illustrated and discussed with respect to FIG. 3. In some embodiments, tubes 10 or 110 are made from a flexible biocompatible polymer, such as but no limited to liquid crystal polymer (LCP), poly(methyl methacrylate) (PMMA), polysulfone (PSU), polyphenylsulfone (PPSU) and the like.

Reference is now made to FIG. 1C which is an illustration of another nonlimiting example of an implantable device according to some embodiments of the invention. An implantable device 250 may include a tube 210 having a first internal lumen 212 comprising one or more microparticles 214 surrounded by first liquid 211. In some embodiments, the diameter of each microcapsule 214 is between 0.5 to 0.9 of the first internal lumen's hydraulic diameter. In some embodiments, device 250 may be dimensioned to be implanted in an organ of a subject. In the nonlimiting example, illustrated in FIG. 1C the organ may be the eye of the subject and therefore, a hydraulic diameter of first inner lumen 112 is between 40 to 150 microns. In some embodiments, the diameter of one or more microparticles 214 is larger than 15 microns, for example, 20 to 134 microns.

In some embodiments, microparticles 214 may be substantially the same as, microparticles 14 and 114 of devices 100 and 200. In some embodiments, a density of microparticles 214 is lower than the density of first liquid 211 by at least 5%, for example, by at least 7%, by at least 10%, by at least 15%, by at least 20%, by at least 30%, by at least 40%, by at least 50% or more. In a nonlimiting example, microparticles 214 may be microcapsules filled with air or any other gas or solution lighter than the liquid, and liquid 211 may be water, hypodermoclysis, any intravenous infusion, silicone-based gel or other viscous biocompatible fluid and the like.

In some embodiments, device 250 may further include a liquid reservoir 220 comprising a flexible cover 222, in fluid connection with a first end 223 of first internal lumen 212. Liquid reservoir 220 is configured to provide a second liquid 213 to internal lumen 212. In some embodiments, first internal lumen 212 may include another portion filled with second liquid 213, the second portion is defined between first liquid 211 and a gas 235 located at end 225 of internal lumen 212.

In some embodiments, first liquid 211 and second liquid 213 are unmixable. For example, first liquid 211 may be water or any other aqueous solution and second liquid 213 may be oil or an oily solution, or vise versa. The structure of device 250 may allow easer detection of the movement of one or more microparticles 214 (e.g., each microparticle 214 separately) along internal tube 212. In some embodiments, one or more microparticles 214 and first liquid 211 move together as a result of a pressure applied on second liquid 213 in liquid reservoir 220. First liquid 211 may be enclosed between two areas of second liquids 213, therefore, one or more microparticles 214 are restricted to move only together with first liquid 211.

In some embodiments, device 250 may further include a gas reservoir 230 in fluid connection to a second end 225 of first internal lumen 212. In the nonlimiting example of FIG. 1C, tube 210 may include a second internal lumen 230 serving as a gas reservoir.

In some embodiments, device 100, 200, and 250 may further include one or more haptic arms 40 extending from tube 110. Haptic arms 40 are configured to support tube 110 inside the organ, for example, in the eye as illustrated in FIG. 2.

In some embodiments, the external device may detect the movement of a single microparticle 14, 114, 214 or a plurality of microparticles 14, 114, 214. The external device (e.g., the camera) may detect the movement of each particle in the plurality of microparticles or of some individual microparticles 14, 114, 214 from the plurality.

Reference is now made to FIG. 2 which is an illustration of an image of an implantable device such as devices 100 implanted in an eye 5 of a user. In the nonlimiting example of FIG. 2, devices 100 and also possibly devices 200 or 250 is implanted in the anterior chamber of eye 5. In some embodiments, the implantation can be incorporated with cataract surgery. As should be understood by one skilled in the art, devices 100 and/or 200 do not include or require any power source, nor need to be powered. Therefore, devices 100 and/or 200 can remain in the eye for several years, for example, 5 to 10 years without any intervention.

Reference is now made to FIG. 3 which is an illustration of another nonlimiting example for an implantable device for detecting in vivo pressure in an organ of a subject according to some embodiments of the invention. A device 300 may be implanted in organs such as a blood vessel (e.g., an artery) or a lunge segment.

An implantable device 300 may include a tube 310 having a first internal lumen 312 comprising microparticles 314. In some embodiments, the diameter of each microcapsule 314 is between 0.5 to 0.9 of the first internal lumen's hydraulic diameter. In some embodiments, device 300 (and therefore tube 310) may be dimensioned to be implanted in an organ of a subject. In the nonlimiting example, illustrated in FIG. 3 the organ may be a blood vessel (e.g., an artery) of the subject and a hydraulic diameter of first inner lumen 312 may be between 150 to 3500 microns. In another nonlimiting example, the organ may be a lung segment of the subject and a hydraulic diameter of first inner lumen 312 is between 120 to 1500 microns. In some embodiments, tube 310 may be made from a flexible material configured to be adjusted to the shape of the blood vessel or the lunge segment.

In some embodiments, a dimeter of each microcapsule 314 is between 60 microns to 3150 microns, or any value in between,

In some embodiments, microparticles 314 may include a magnetic material or an electrically conductive material configured to be detected by a sensor capable of detecting such materials. For example, microparticles 314 may include metallic powder, carbon powder, ceramic material, and the like.

In some embodiments, device 300 may further include a liquid reservoir 320 comprising a flexible cover 322, in fluid connection with a first end 313 of first internal lumen 312, for providing liquid 324 for surrounding microparticles 314. In some embodiments, a density of microparticles 314 is lower than the density of liquid 324 by at least 5%, for example, by at least 7%, by at least 10%, by at least 15%, by at least 20%, by at least 30%, by at least 40%, by at least 50% or more. In a nonlimiting example, microparticles 314 may be microcapsules filled with air or any other gas or solution lighter than the liquid, and liquid 324 may be water, hypodermoclysis, any intravenous infusion, silicone-based gel or other viscous biocompatible fluid and the like.

In some embodiments, a material of flexible cover 322 is selected such that a pressure applied on flexible cover 322 (e.g., the blood pressure), after the implantation, causes a flow of liquid 324 from liquid reservoir 320 into first internal lumen 312. In the nonlimiting example of FIG. 3, liquid reservoir 320 is a balloon and the flexible cover 322 is included in the outer shell of the balloon.

In some embodiments, device 300 may further include a gas reservoir 330 in fluid connection to a second end 315 of first internal lumen 312. In the nonlimiting example of FIG. 3 tube 310 may include a second internal lumen 330 being the gas reservoir.

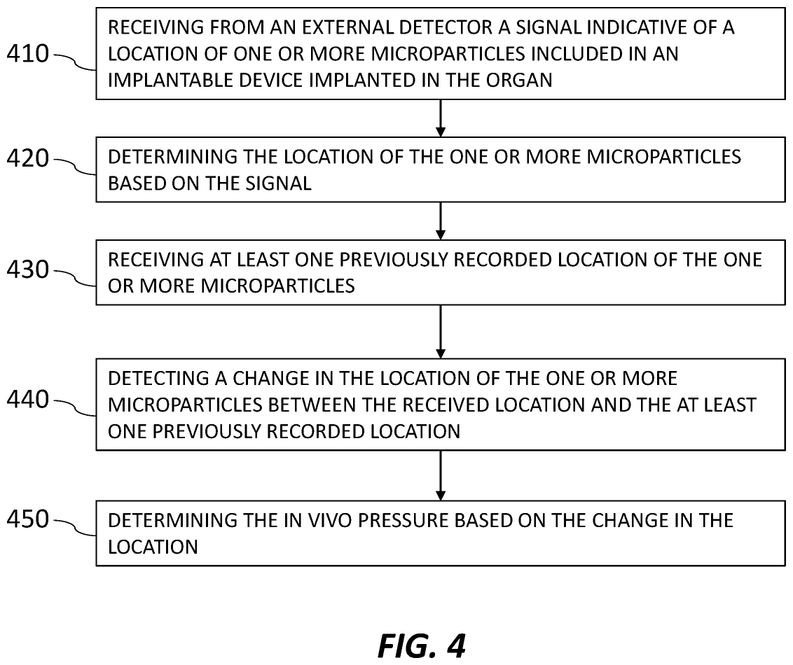

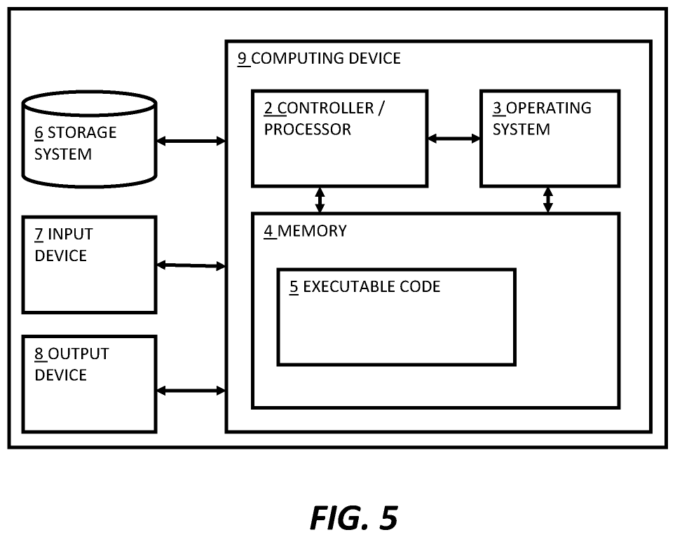

Reference is now made to FIG. 4 which is a flowchart of a method of determining in vivo pressure in an organ of a subject according to some embodiments of the invention. The method of FIG. 4 may be executed by computing device 10 illustrated and discussed with respect to FIG. 5 or by any other computing device. In some embodiments, the method may be performed following an implant of a device such as, device 100, 200, 250, or 300 in the organ of the subject.

In step 410, a signal may be received from an external detector, the signal is indicative of a location of one or more microparticles included in an implantable device implanted in the organ. For example, the signal may be an image of an eye that shows microparticles 14, 114, or 214. In another example, the signal may be a thermal image of an arm showing metallic microparticles 340 inside a blood vessel. In yet another example, the image can be an X-ray image of the chest showing metallic/magnetic microparticles 340 in a lunge segment. In some embodiments, at least one microparticle is detectable in the image, for example, a single microparticle, or a plurality of distinctive microparticles, each of these microparticles can be separately detected in the image.

In step 420, the location of the one or more microparticles may be determined based on the signal. In a nonlimiting example, determining the location may include, receiving at least two known locations (e.g., three known locations) in the implantable device, for example, the locations of first end 13/113/223/313 and second end 15/115/225/315 (and optimally an additional location in-between). The method may include, identifying the at least two known locations in the signal, for example, in the image, the thermal image, or the X-Ray image, identifying the microparticles in the signal, and calculating the relative location of the microparticles with respect to the two known locations. For example, any image analysis program may be used for identifying first end 13/113/313 and second end 15/115/315 and particles 14/114/314 in any type of image. In the nonlimiting examples of the optical image first end 13/113 and second end 15/115 may be marked with a florescent material or a specific color. In the nonlimiting examples of the thermal and/or X-Ray images, first end 313 and second end 315 may be marked with a conductive or magnetic marker. Accordingly, the location of the microparticles may be determined based on the signal indicative of the location of the microparticles and the at least two known locations.

In step 430, at least one previously recorded location of the microparticles may be received, for example, from storage system 6 of commuting device 9. The previously recorded location may be determined in a similar manner to the determination discussed in step 420, using signals received from the same external detector, detecting the same implantable device implanted in the same organ.

In step 440, a change is detected, in the location of the microparticles (each microparticle separately) between the received location and the at least one previously recorded location. The change may indicate movement of the microparticles due to a change in an in vivo pressure in the organ pressing flexible cover 22/122/322 thus causing liquid 24/124/324 to flow from liquid reservoir 20/120/320 to tube 10/110/310 while pushing microparticles 14/114/314 to move along first lumen 12/112/312 or vice versa. The higher the in vivo pressure, the larger is the detected change. Therefore, in step 450, the in vivo pressure may be determined based on the change in the location. In some embodiments, a lookup table may be stored in storage system 6, the lookup table may associate changes in a location with levels of in vivo pressure.

In a nonlimiting example, the external detector is an optical camera, and the method comprises: receiving from the optical camera, for example, optical camera included in user device 80, an image of eye 5 that includes an image of the microparticles 14 or 114; determining a location of the microparticles in internal lumen 12 or 112 from the image. The method may further include receiving at least one previously recorded location of the microparticles in the internal lumen; and detecting intraocular pressure if the change in the location is greater than a threshold value.

In another nonlimiting example, the external detector is a thermal camera, that may be included in user device 80 or may be a dedicated device, and the in vivo pressure may be the blood pressure of the subject.

In another nonlimiting example, the external detector is an X-Ray camera, MRI or CT devices and the in vivo pressure may be pressure in a lunge segment.

Reference is now made to FIG. 5, which is a block diagram depicting a computing device, which may be included within an embodiment of a system for detecting in vivo pressure in an organ of a subject, according to some embodiments

Computing device 9 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 9 may be included in, and one or more computing devices 9 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 10, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random Access Memory (RAM), a read-only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task or script. Executable code 5 may be executed by processor or controller 2 possibly under control of operating system 3. For example, executable code 5 may be an application that may include a method for detecting in vivo pressure in an organ of a subject as described herein above. Although, for the sake of clarity, a single item of executable code 5 is shown in FIG. 5, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Previously recorded location of the microparticles of devices 100, 200 and 300 may be stored in storage system 6 and may be loaded from storage system 6 into memory 4 where it may be processed by processor or controller 2. In some embodiments, some of the components shown in FIG. 5 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to computing device 9 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. An implantable device, comprising:
a tube having a first internal lumen comprising one or more microparticles surrounded by a first liquid;
a liquid reservoir comprising a flexible cover, in fluid connection with a first end of the first internal lumen, for providing a second liquid to the first internal lumen;
a gas reservoir in fluid connection with a second end of the first internal lumen;
wherein each microparticle has a diameter between 0.5 to 0.9 of the first internal lumen's hydraulic diameter and comprises a material allowing a detection of a movement of each microparticle separately by an external device.

2. The implantable device of claim 1, wherein the first liquid and the second liquid are the same liquid.

3. The implantable device of claim 1, wherein the first liquid and the second liquid are unmixable.

4. The implantable device of claim 1, wherein a density of the microparticles is lower than a density of the first liquid by at least 5%.

5. The implantable device of claim 1, wherein the tube is curved or straight.

6. The implantable device of claim 1, wherein the tube is made from a flexible material.

7. The implantable device of claim 1, wherein
the microparticles include a material visible in an image taken by an optical camera.

8. (canceled)

9. (canceled)

10. The implantable device of claim 1, wherein the microparticles include a magnetic material and/or an electrically conductive material.

11. The implantable device of claim 1, wherein a diameter of each microparticle is larger than 15 micron.

12. The implantable device of claim 1, wherein the liquid reservoir is a balloon and wherein the flexible cover is included in the outer shell of the balloon.

13. The implantable device of claim 10, further comprising a second internal lumen being the gas reservoir.

14. The implantable device of claim 1, wherein the liquid reservoir has a flat shape with an open face covered by a flexible membrane.

15. The implantable device of claim 14, wherein the gas reservoir has a flat shape.

16. The implantable device of claim 1, wherein the organ is an eye of the subject and wherein the hydraulic diameter of the first inner lumen is between 40 to 150 microns.

17. The implantable device of claim 1, wherein the organ is a blood vessel of the subject and wherein the hydraulic diameter of the first inner lumen is between 150 to 3500 microns.

18. The implantable device of claim 1, wherein the organ is a lung segment of the subject and wherein the hydraulic diameter of the first inner lumen is between 120 to 1500 microns.

19. The implantable device of claim 1, wherein a material of the flexible cover is selected such that a pressure applied on the flexible cover, after the implantation, causes a flow of liquid from the liquid reservoir into the first internal lumen.

20. The implantable device of claim 1, wherein the microparticles are microcapsules.

21. A method of determining in vivo pressure in an organ of a subject, comprising:
receiving from an external detector a signal indicative of a location of one or more microparticles included in an implantable device implanted in the organ;
determining the location of the one or more microparticles based on the signal;
receiving at least one previously recorded location of the one or more microparticles;
detecting a change in the location of the one or more microparticles between the received location and the at least one previously recorded location; and
determining the in vivo pressure based on the change in the location.

22. The method of claim 21, wherein determining the location of the one or more microparticles comprises:
receiving at least two known locations in the implantable device;
identifying the at least two known locations in the received signal; and
determining the location of the one or more microparticles based on the signal indicative of the location of the one or more microparticles and the at least two signals.

23.-27. (canceled)
